(19) United States
(12) Plant Patent Application Publication
(10) Pub. No.: US 2015/0156935 P1
(43) Pub. Date: Jun. 4, 2015

(54) Pistachio Rootstock Tree Named Paramount Pistachio Rootstock #1

(50) ;

(71) Applicant: Paramount Farming Company, Bakersfield, CA (US)

(72) Inventors: Joceph Macllvaine, Bakersfield, CA (US); Eric Mercura, Bakersfield, CA (US); John Etchamendy, Wasco, CA (US)

(73) Assignee: Paramount Farming Company, Bakersfield, CA (US)

(21) Appl. No.: 13/998,771

(22) Filed: Dec. 4, 2013

(51) Int. Cl. A01H 5/00

(52) U.S. Cl. PLT/152

(57) ABSTRACT

A new and distinct variety of pistachio tree characterized by producing large leaves, reddish-green new foliage, and dark green mature foliage. The new variety is useful as a rootstock for commercial nut bearing pistachio varieties.

### LATIN NAME OF THE GENUS AND SPECIES CLAIMED

Pistacia atlantica×integerrima

### VARIETY DENOMINATION

PARAMOUNT PISTACHIO ROOTSTOCK #1

### BACKGROUND AND SUMMARY OF THE INVENTION

This invention relates to the discovery and asexual propagation of a new and distinct variety of Pistachio tree, Pistachio atlantica×integerrima, cv. Paramount Pistachio Rootstock #1 as herein described and illustrated. The new variety is a hybrid of P. atlantica and P. integerrima which was first discovered on cultivated land from trees grown from seed produced by a cross of the two parent varieties. The new variety was discovered and further characterized, and propagated by Joseph Macllvaine, Eric Mercure and John Etchamendy in the San Joaquin Valley, Kern County, Calif. The initial discovery, characterization and propagation of the new variety took place from 2006 to 2010.

The new variety Paramount Pistachio Rootstock #1 is characterized by producing large leaves, reddish-green new foliage, dark green mature foliage and upright habits with rounded crown. The new variety is useful as a rootstock to nut bearing pistachio.

The new variety Paramount Pistachio Rootstock #1 was first asexually propagated in 2002 by Eric Mercure by tissue culture at Southern San Joaquin Valley in Ken County, Calif. and established and transmitted through succeeding propagation by air-layering.

The male parent is P. integerrima and the female parent is P. atlantica. The date of first sowing was 1990, and the date of first flowering was 1996.

The new variety Paramount Pistachio Rootstock #1 differs from its male parent P. integerrima in that the leaves and leaflets are shorter. Also, the leaflet base is more rounded in the new variety. In addition, the leaves are less red when emerging and fall off sooner.

The new variety Paramount Pistachio Rootstock #1 differs from its female parent P. atlantica in that new variety is more vigorous and is believed to be more resistant to verticillium disease. The leaflets of the new variety are smaller and less rounded with a faster growth rate. The new variety has an overall larger size canopy.

The new variety can be distinguished from other available varieties by its yield characteristics. The closest available varieties are Integerrima “Pioneer Gold” and UCB1, an atlantica×integerrima hybrid. Yields on P. vera have been observed to be consistently higher on mature trees on the new rootstock variety when compare to Pioneer Gold and UCB1. The new variety Paramount Pistachio Rootstock #1 can also be distinguished from “Pioneer Gold” by the leaf shape.

The new Paramount Pistachio Rootstock #1 has been shown to maintain its distinguishing characteristics through successive asexual propagations by, for example, tissue culture and air layering.

Variations of the usual magnitude from the characteristics described herein may occur with changes in growing conditions, irrigation, fertilization, pruning, management and with climatic variation.

The new variety Paramount Pistachio Rootstock #1 was analyzed to identify unique molecular markers. In one set of experiments primers were used to amplify a variety of simple sequence repeats (SSRs), essentially as described in Vendramin et al. Mol. Ecology Res. (2010) 10(3):576-579. SSRs were identified from a GT-enriched library prepared from P. atlantica and P. integerrima DNA and from the literature (Vendramin et al., Ahmad et al. J. Amer. Soc. Hort. Sci. (2003) 128(6):898-903 and Albaladejo et al. Mol. Ecology Res. (2008) 8:904-906). Primers were prepared and used to amplify relevant loci. Paramount Pistachio Rootstock #1 was compared to its parents, P. integerrima and P. atlantica, as well as UCB1 and two additional unpatented varieties. Paramount Pistachio Rootstock #1 was uniquely identified by products at about 625 and 655 by using a primer set corresponding to EPV F013 from Vendramin et al.

DNA amplification fingerprinting (DAF) was also used to analyze Paramount Pistachio Rootstock #1, essentially as described in Caetano-Anolles BioTechniques (1998) 25:472-480 and Trigiano, R. N. and G. Caetano-Anolles. HortTechnology (1998) 8(3):413-423. Two DAF primers, 8.6D and 8.6J (Trigiano, R. N. et al. HortTechnology (2004) 39(3):489-492) were able to distinguish Paramount Pistachio Rootstock #1 from UCB1 and the two unpatented varieties, as well as from P. integerrima and P. atlantica. Primer 8.6D produced a doublet at about 410 bp that was only present in Paramount Pistachio Rootstock #1 and UCB1. The doublet was not present in the parent varieties or in the unpatented varieties. Primer 8.6J was then able to distinguish Paramount Pistachio Rootstock #1 from UCB1, as it produced heavy bands at 150-200bp in Paramount Pistachio Rootstock #1. These bands were not present for UCB1 or for the unpatented varieties.

### BRIEF DESCRIPTION OF THE DRAWINGS

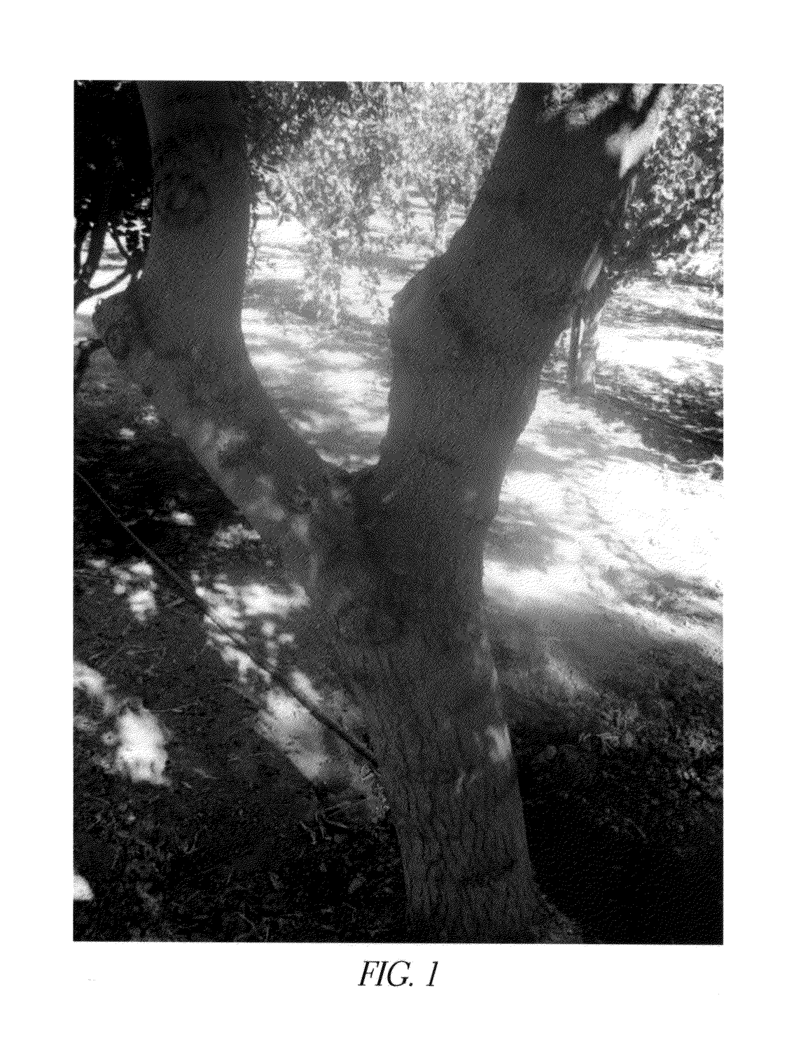

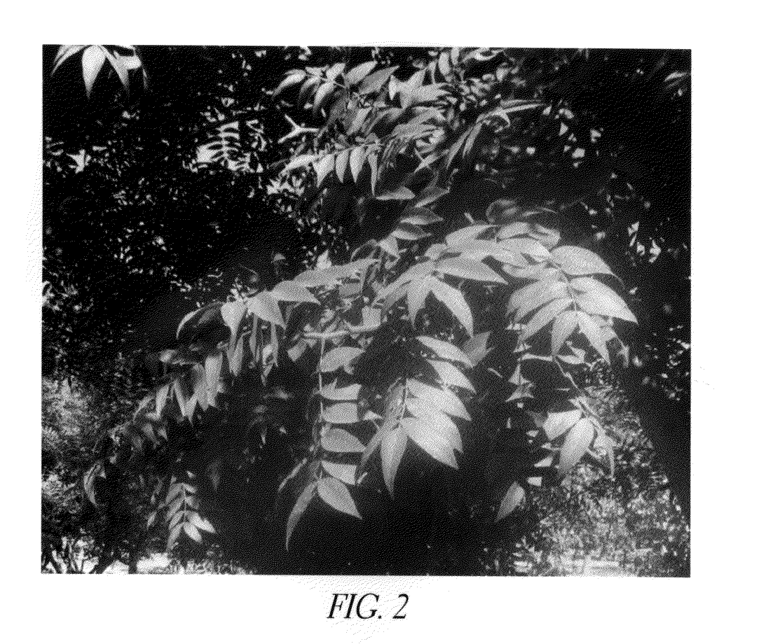

The accompanying color photographic illustration FIG. 1 shows a mature tree with foliage of the new variety Paramount Pistachio Rootstock #1. Additionally, FIG. 2 shows a close up of the foliage. The colors of photographic illustration are as nearly true as is reasonably possible in a color representation of this type.

### DETAILED DESCRIPTION OF THE PREFERRED EMBODIMENT

Throughout this specification, color names beginning with a small letter signify that the name of that color, as used in common speech, is aptly descriptive. Color names beginning with a capital letter designate values based upon the R.H.S. Colour Chart, published by The Royal Horticultural Society, London, England, 5^{th} edition, 2007.

The descriptive matter which follows pertains to ‘Paramount Pistachio Rootstock #1’ grown in the vicinity of the Southern San Joaquin Valley in Ken County, Calif. during 2002 to present, and is believed to apply to plants of the variety grown under similar conditions of soil and climate elsewhere.
Plant:
   General.—Growth: Upright tree with a rounded crown reaching 70-100 feet in height.
Foliage:
   Leaves.—Number of leaflets: 7-9. Average length: Long, approximately 8-10 inches. Average Width: Medium, approximately 5-6 inches.
   Leaflets.—Size: Medium, approximately 3 inches Shape: Lanceolate. Length: Medium, approximately 2.5-3 inches. Color of new leaflets: About Dark Greyed-purple 183B Color of old leaflets: About Dark Green 137A, Dark Green 137B and Yellow-Green 147B Edge: Entire. Petiole: Petiolated. Arrangement of Petiole: Pinnately compound. Arrangement of Stems: Opposite. Shape of leaf at Apex: Acuminate. Shape of leaf at Base: Acute. Margin: Entire. Venation: Pinnate. Vein Color: About Dark Green 137A, Dark Green 137B and Yellow-Green 147B Texture of top surface: Globrous. Texture of bottom of surface: Globrous.
   Ribs.—Length: Approximately 6-8 inches Color. About Dark Greyed-Purple 183A-B to Medium Yellow-Green 144A-B, and underside color About Medium Yellow-Green 144B.
   Petiole.—Length: Approximately 2 inches Color. About Dark Greyed-Purple 183B to Medium Yellow-Green 144A-B Diameter. Less than ⅛ inch.
   Growth.—Trunk shape: Upright Trunk color. About Dark Brown 200B with fissures About Medium Grey-Brown 199D. Trunk diameter. Large. Bark texture: Striated when young and slightly furrowed with age. Vegetative bud size: approximately ⅛″-¼″ Vegetative bud color: reddish-gray.

What is claimed is:

1. A new and distinct variety of pistachio tree as herein illustrated and described.
